(19) United States
(12) Patent Application Publication
(10) Pub. No.: US 2025/0163437 A1
(43) Pub. Date: May 22, 2025

(54) BACTERIA-FREE APPROACH FOR EXPRESSING NUCLEIC ACIDS AND PROTEINS IN EUKARYOTIC CELLS

(71) Applicant: Clemson University Research Foundation, Clemson, SC (US)

(72) Inventors: Andrei Alexandrov, Clemson, SC (US); Rui Che, Greenwood, SC (US); Bhoomi Mirani, Greenwood, SC (US); Monireh Mohammadpanah, Greenwood, SC (US)

(21) Appl. No.: 18/949,053

(22) Filed: Nov. 15, 2024

Related U.S. Application Data
(60) Provisional application No. 63/599,852, filed on Nov. 16, 2023.

(51) Int. Cl. C12N 15/66; C12N 15/10; C12N 15/85

(52) U.S. Cl. CPC C12N 15/66; C12N 15/10; C12N 15/85; C12N 2310/315

(57) ABSTRACT

This invention is directed to methods and compositions for bacteria-free preparation, assembly and expression of heterologous products in eukaryotic cells, including an in vitro method for joining ends of one or more linear double-stranded nucleic acid molecules to form a circular double-stranded nucleic acid construct for use in transfection, electroporation, nucleofection, and/or other form of delivery of the circular nucleic acid construct into a eukaryotic cell, as well as methods for making and compositions comprising the nucleic acid molecules and nucleic acid constructs for use in the methods disclosed herein.

### PRIORITY STATEMENT

This application claims the benefit, under 35 U.S.C. § 119 (e), of U.S. Provisional Application Ser. No. 63/599,852, filed Nov. 16, 2023, the entire contents of which are incorporated by reference herein.

### STATEMENT OF GOVERNMENT SUPPORT

This invention was made with government support under Grant Number GM139769 awarded by the National Institutes of Health. The government has certain rights in the invention.

### FIELD OF THE INVENTION

This invention is directed to methods and compositions for bacteria-free preparation, assembly and expression of heterologous products in eukaryotic cells.

### BACKGROUND OF THE INVENTION

Current commercial DNA assembly techniques and plasmid generation techniques are laborious and time-consuming not least due to their required involvement of bacterial-dependent steps. These required steps lead to the need for various time consuming additional steps and components including the need for electroporators, bacterial incubators, bacterial shakers, refrigerated centrifuges, vacuum manifolds, plasmid preps, bacterial competent cells, and the like, leading to an average time requirement of over two days for the production of efficient yields of product for transfection into human cells. However, up to date there have been no viable alternatives to E. coli or other bacterial-based methods to allow for efficient transfer and expression of product in eukaryotic cells.

The present invention overcomes previous shortcomings in the art by providing methods of bacteria-free assembly of nucleic acid molecules for direct transfer and expression of products in eukaryotic cells.

### SUMMARY OF THE INVENTION

One aspect of the present invention provides an in vitro method for joining ends of one or more linear double-stranded nucleic acid molecules to form a circular double-stranded nucleic acid construct for use in transfection, electroporation, nucleofection, and/or other form of delivery of the circular nucleic acid construct into a eukaryotic cell, comprising: joining the ends of the one or more linear nucleic acid molecules in a single reaction comprising the following mixture of components (a) to (d): (a) the one or more linear double-stranded nucleic acid molecules to be joined, wherein each of nucleic acid molecules to be joined comprises ends (e.g., a 5′ end and a 3′ end) comprising overlapping terminal sequences flanked by one or more nucleotides connected via a modified phosphodiester group, (b) a 5′ to 3′ exonuclease that lacks the 3′ to 5′ exonuclease activity and whose 5′ to 3′ exonuclease hydrolysis activity is reduced by the modified phosphodiester group of each of the linear double-stranded nucleic acid molecules, (c) a DNA ligase, and (d) a buffer suitable for joining ends of the said one or more linear double-stranded nucleic acid molecules using concomitant activity of the 5′ to 3′ exonuclease and the DNA ligase in the single reaction; wherein at least one of the one or more linear nucleic acid molecules comprises and/or encodes a product for expression in the eukaryotic cell, and wherein the product is expressed in the eukaryotic cell upon delivery without the requirement to transform and/or propagate the circular nucleic acid construct in bacteria at any step of the method.

Another aspect of the present invention provides a method of producing a synthetic linear double-stranded nucleic acid molecule comprising overlapping terminal sequences flanked by one or more nucleotides connected via a modified phosphodiester group (e.g., generating a linear double-stranded nucleic acid molecule for use in the methods of the invention), comprising: amplifying a synthetic linear double-stranded nucleic acid molecule comprising an RNA polymerase terminator sequence, RNA cleavage and polyadenylation sequence, and/or an RNA scaffold followed in 5′ to 3′ orientation by a promoter and/or an enhancer, to produce a population of synthetic linear double-stranded nucleic acid molecules under conditions in which each of the amplified linear double-stranded nucleic acid molecule generated comprises the overlapping terminal sequences flanked by the one or more nucleotides connected via a modified phosphodiester group.

Another aspect of the present invention provides a synthetic double-stranded nucleic acid molecule comprising an RNA polymerase terminator sequence, RNA cleavage and polyadenylation sequence, and/or an RNA scaffold followed in 5′ to 3′ orientation by a promoter and/or an enhancer (e.g., a “nano-template”). In some embodiments, a synthetic double-stranded nucleic acid molecule comprising an RNA polymerase terminator sequence, RNA cleavage and polyadenylation sequence, and/or an RNA scaffold followed in 5′ to 3′ orientation by a promoter and/or an enhancer of the invention may be generated by a method as described herein and/or used in a method described herein.

Another aspect of the present invention provides a synthetic linear double-stranded nucleic acid molecule (e.g., “nano-backbone”) comprising: an RNA polymerase terminator sequence, RNA cleavage and polyadenylation sequence, and/or an RNA scaffold followed in 5′ to 3′ orientation by a promoter and/or an enhancer, and overlapping terminal sequences flanked by the one or more nucleotides connected via a modified phosphodiester group. In some embodiments, a synthetic linear double-stranded nucleic acid molecule comprising an RNA polymerase terminator sequence, RNA cleavage and polyadenylation sequence, and/or an RNA scaffold followed in 5′ to 3′ orientation by a promoter and/or an enhancer, and overlapping terminal sequences flanked by the one or more nucleotides connected via a modified phosphodiester group may be generated by a method as described herein and/or used in a method described herein.

### BRIEF DESCRIPTION OF THE DRAWINGS

FIG. 1 shows an example illustrative schematic of the generalized steps of the methods of the invention.

FIG. 2 shows illustrative schematics and data plots regarding the methods of the invention. FIG. 2 panel A shows an illustrative schematic of an example reaction workflow. FIG. 2 panel B shows an image of an agarose gel analysis of the product of the reaction as schematized in FIG. 2 panel A. (1) 100 bp DNA ladder; (2) insert; (3) backbone; (4) resulting nanocircle (EcoRI-linearized to show size): and (5) EcoRI-cleaved backbone. FIG. 2 panel C shows images of FACS data plots analyzing fluorescence produced by 2 μg of pMaxGFP plasmid (left plot) and 2 μg of GFP nanocircles produced by the methods of the invention (middle panel) after transfection into HEK293T cells. Transfection of nanocircles results in higher fluorescence due to smaller size. FIG. 2 panel D shows further FACS analysis plots showing transfection of sgRNA-expressing nanocircles produced by the methods of the invention produce efficient knockout of the nonsense-mediated mRNA degradation (NMD) factor CASC3 in Cas9-expressing Fireworks HeLa cells.

FIG. 3 shows images of fluorescence microscopy indicating efficient transfection and expression of fluorescent proteins by enzymatically-produced DNA nanocircles of the invention in human cells. Each nanocircle was produced in a 30-minute reaction in which an open reading frame of a fluorescent protein was inserted into the nanobackbone, placing fluorescent protein under control of the CMV promoter and followed by a Bovine Growth Hormone (BGH) cleavage and polyadenylation sequence. Following a brief 10-minute purification of the reaction products using Promega Wizard Gel and PCR Clean-up kit, each well of a 6-well plate of adherent Human Embryonic Kidney cells (HEK293T) was transfected with 2.5 μg of nanocircles using Mirus 293 transfection reagent. Images were taken 48 hours post-transfection using Olympus IX70 fluorescence microscope using 0.25, 0.25, 0.5, and 0.04 second exposure time for mCherry, tdTomato, eGFP, and pMaxGFP-expressing nanocircles, respectively. Each fluorescence image is accompanied with a phase-contrast image that is shown below the fluorescence image to indicate cell density.

FIG. 4 shows images of microscopy indicating efficient expression of functionally-active antibiotic selection markers by enzymatically-produced DNA nanocircles of the invention in human cells. Each nanocircle was produced in a 30-minute reaction in which an open reading frame of an antibiotic resistance marker was inserted into the nanobackbone, placing antibiotic resistance marker under control of the short EF1alfa promoter (EFS) and followed by a Bovine Growth Hormone (BGH) cleavage and polyadenylation sequence. Following a brief 10-minute purification of the reaction products using Promega Wizard Gel and PCR Clean-up kit, each well of a 6-well plate of adherent Human Embryonic Kidney cells (HEK293T) was transfected with 3.0 μg of nanocircles (left column) or bacteria-produced plasmids (middle column) carrying identical antibiotic-expressing cassettes (promoter, antibiotic selection marker, and cleavage polyadenylation sequence) using Mirus 293 transfection reagent. Cells were grown in DMEM media in the presence of antibiotic at the concentrations indicated in the figure. Phase contrast images were taken 2, 2, 2, and 4 days post-transfection for puromycin, blasticidin, hygromycin, and neomycin resistance markers, respectively using Olympus IX70 microscope to analyze cell density as a measure of cell survival under antibiotic selection pressure.

FIG. 5 shows a more detailed exemplar schematic of the workflow of the methods disclosed herein.

FIG. 6 shows a schematic of a non-limiting example of a nanocircle of the invention and well as non-limiting optional size parameters, promoter options, and polyA and/or RNA scaffold options.

FIG. 7 shows a schematic of a non-limiting example of a nanobackbone and options for structural components, a nanotemplate and options for structural components, and a generalized schematic of a linear nanobackbone PCR product.

FIG. 8 shows a schematic of an example reaction mixture, nanotemplate and ready-to-use linear nanobackbone. Also listed are non-limiting examples of generated nanotemplate and nanobackbone constructs with variable promoter and 3′ site (transcription terminator, cleavage and polyadenylation sequence, or RNA scaffold) options generated.

FIG. 9 shows a schematic diagram and an image of gel analysis confirming an embodiment of circular construct assembly of the invention.

FIG. 10 shows a schematic diagram and an image of gel analysis confirming an embodiment of circular construct assembly of the invention.

FIG. 11 shows a schematic diagram and an image of gel analysis confirming an embodiment of linear construct assembly of the invention.

FIG. 12 shows histogram plots indicating successful knockdowns of GFP and RFP using transfected nanocircles versus E. coli-obtained (pLKO.1) plasmids.

FIG. 13 shows histogram plots indicating successful knockout of GFP and RFP using transfected nanocircles versus E. coli-obtained (LtCRISPR) plasmids.

FIG. 14 shows images of fluorescence microscopy and histogram plots relating to Fluorescence of nanocircles versus that of E. coli-obtained plasmids.

FIG. 15 shows images of fluorescence microscopy and histogram plots relating to Expression of GFP in human HEK293T cells using nanocircles with different promoters.

FIG. 16 shows a diagram of sequencing data indicating that lentiviral sgRNA libraries produced via the methods of the invention (e.g., no E. coli at any step) are free from negative effects of the E. coli transformation bottleneck.

FIG. 17 shows images of histograms indicating that inhibition of RNase P activity in the human MALAT1 Mirror HeLa cell line using lentiviruses produced by the methods of the invention, expressing sgRNA targeting RNase P component RPP21.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention. Where the stated range includes one or both of the limits, ranges excluding either or both of those included limits are also included in the invention.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “subject” and “patient” are used interchangeably herein and refer to both human and nonhuman animals. A subject of this invention can be any subject that is susceptible to a disorder that can benefit by the methods and compositions of the present invention and/or be treated for a disorder by the methods and compositions of the present invention. In some embodiments, the subject of any of the methods of the present invention is a mammal. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. As a further option, the subject can be a laboratory animal and/or an animal model of disease. In some preferred embodiments, the subject is a human. The subject may be of any gender, any ethnicity and any age.

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art. Thus, all or a portion of the nucleic acids of the present codons may be synthesized using codons preferred by a selected host. Species-preferred codons may be determined, for example, from the codons used most frequently in the proteins expressed in a particular host species. Other modifications of the nucleotide sequences may result in mutants having slightly altered activity.

As used herein, the term “polypeptide” encompasses both peptides and proteins (including fusion proteins), unless indicated otherwise.

As used herein with respect to nucleic acids, the term “operably linked” refers to a functional linkage between two or more nucleic acids. For example, a promoter sequence may be described as being “operably linked” to a heterologous nucleic acid sequence because the promoter sequence initiates and/or mediates transcription of the heterologous nucleic acid sequence. In some embodiments, the operably linked nucleic acid sequences are contiguous and/or are in the same reading frame.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of a source material from which the polynucleotide is isolated, including but not limited to an in vitro mixture, or a naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of a source material from which the polypeptide is isolated, including but not limited to an in vitro mixture, or the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a source sequence due to one or more deletions, additions, substitutions, or any combination thereof. Modified sequences may also be referred to as “modified variant(s).”

A “vector” or “construct” as used herein refers to a compound used as a vehicle to carry foreign genetic material into another cell, where it can be replicated and/or expressed. A vector or construct containing foreign nucleic acid may be termed a recombinant vector or recombinant construct (e.g., expression construct). Examples of nucleic acid vectors or nucleic acid constructs are plasmids, viral vectors, cosmids, expression cassettes, and artificial chromosomes. In the current state of the art, recombinant vectors typically contain an origin of replication, a multicloning site, and a selectable marker. The nucleic acid sequence typically consists of an insert (recombinant nucleic acid or transgene) and a larger sequence that serves as the “backbone” of the construct. The purpose of a construct which transfers genetic information to another cell is typically to isolate, multiply, or express the insert in the target cell. Expression vectors (expression constructs or expression cassettes) are for the expression of the exogenous gene and/or gene product in the target cell, and generally have a promoter sequence that drives expression of the exogenous gene and/or gene product. Insertion of a vector or construct into the target cell is referred to transformation or transfection for bacterial and eukaryotic cells, although insertion of a viral vector is often called transduction. The term “vector” or “construct” may also be used in general to describe items to that serve to carry foreign genetic material into another cell, such as, but not limited to, a transformed cell or a nanoparticle.

As used herein, by “isolate” or “purify” (or grammatical equivalents) a vector or construct, it is meant that the vector or construct is at least partially separated from at least some of the other components in the starting material.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold, and/or at least about 5%, 10%, 15%, 20%, 25%, 30%, 35%, 40%, 45%, 50%, 55%, 60%, 65%, 70%, 75%, 80%, 85%, 90%, 95%, or 100% or more, or any value or range therein.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts may be referred to as “transcription products” and encoded polypeptides may be referred to as “translation products.” Transcripts and encoded polypeptides may be collectively referred to as “gene products.” If the polynucleotide is derived from genomic DNA, expression may include splicing of the mRNA in a eukaryotic cell. The expression product itself, e.g., the resulting nucleic acid or protein, may also be said to be “expressed.” An expression product can be characterized as intracellular, extracellular, or secreted. The term “intracellular” means something that is inside a cell. The term “extracellular” means something that is outside a cell. A substance is “secreted” by a cell if it appears in significant measure outside the cell, from somewhere on or inside the cell.

A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art.

In those methods of the invention that are carried out “in vitro”, all of the protein components are isolated and/or substantially purified. The in vitro assembly reactions are not carried out in a living cell or with a crude cell extract; the reactions are carried out in a cell-free environment.

The “joining” of the nucleic acid molecules by a method of the invention is sometimes referred to herein as “recombination” or “assembly” of the nucleic acid molecules.

As used herein, the term “minicircles” refers to circular nucleic acid molecules (e.g., double-stranded DNA molecules also referred to as DNA minicircles) which are devoid of bacterial DNA sequence elements such as bacterial antibiotic resistance marker(s) and/or bacterial origin(s) of replication.

As used herein, the term “nano-circle” or “nanocircle” refers to a circular double-stranded nucleic acid molecule, such as but not limited to a DNA molecule. In some embodiments, a nanocircle may be constructed using the methods as described herein.

As used herein, the term “nano-backbone” or “nanobackbone” refers to a linear double-stranded nucleic acid molecule, such as but not limited to a DNA molecule, that comprises precisely positioned phosphorothioate modifications and terminal homology regions. In some embodiments, a nanobackbone of the invention may be used to construct a nanocircle of the invention, for example by the methods as described herein.

As used herein, the term “nano-template” or “nanotemplate” refers to a nucleic acid molecule template, such as but not limited to a DNA template, for amplification reactions such as but not limited to polymerase chain reaction (PCR) amplification, that yield nanobackbones.

In some embodiments, a nanotemplate of the invention may be used to generate a nanobackbone for use in the methods for constructing nanocircles as described herein.

### Methods

The present invention is based at least in part on the novel engineering of a method for assembly of nucleic acid molecules into nanocircles at sufficiently high yield such that the nanocircles may be directly transferred to a eukaryotic cell for expression of any produced and/or encoded product (e.g., gene or gene product), without the requirement for intervening bacterial cloning and/or other amplification steps.

The inventors discovered that nucleic acid molecules modified to comprise phosphorothioate modifications can be joined in a single reaction by the methods as disclosed herein to a highly advantageous efficiency and can produce and express any comprised and/or encoded product directly upon delivery to a human cell, without any bacterial cloning or other amplification or endotoxin clean-up steps as necessary in commercially available DNA assembly and cloning techniques. The nanocircles of the invention may contain DNA nick(s), defined as the absence of a phosphodiester bond between adjacent nucleotides on one strand of a double-stranded DNA molecule. Nano-circles may also contain bacteria-derived sequences including (i) origins of replication and/or (ii) antibiotic selection markers, however, these bacteria-derived sequences are not required for constructing nanocircles using the methods described herein.

Accordingly, one aspect of the present invention provides an in vitro method for joining ends of one or more linear double-stranded nucleic acid molecules to form a circular double-stranded nucleic acid construct for use in transfection, electroporation, nucleofection, and/or other form of delivery of the circular nucleic acid construct into a eukaryotic cell, comprising: joining the ends of the one or more linear nucleic acid molecules in a single reaction comprising the following mixture of components (a) to (d): (a) the one or more linear double-stranded nucleic acid molecules to be joined, wherein each of nucleic acid molecules to be joined comprises ends (e.g., a 5′ end and a 3′ end) comprising overlapping terminal sequences flanked by one or more nucleotides connected via a modified phosphodiester group, (b) a 5′ to 3′ exonuclease that lacks the 3′ to 5′ exonuclease activity and whose 5′ to 3′ exonuclease hydrolysis activity is reduced by the modified phosphodiester group of each of the linear double-stranded nucleic acid molecules, (c) a DNA ligase, and (d) a buffer suitable for joining the ends of said one or more linear double-stranded nucleic acid molecules using concomitant activity of the 5′ to 3′ exonuclease and the DNA ligase in the single reaction; wherein at least one of the one or more linear nucleic acid molecules comprises and/or encodes a product (e.g., a polypeptide, protein, RNP, mRNA, miRNA, shRNA, antisense oligonucleotides (AON; e.g., asRNA), dsRNA, microRNA duplex, pre-miRNA, siRNA, MOA, or the like) for expression in the eukaryotic cell, and wherein the product is expressed in the eukaryotic cell upon delivery without the requirement to transform and/or propagate the circular nucleic acid construct in bacteria at any step of the method. In some embodiments, the 5′ to 3′ exonuclease and the DNA ligase may act concurrently. In some embodiments, the 5′ to 3′ exonuclease and the DNA ligase may act sequentially.

The in vitro method for joining ends of one or more linear double-stranded nucleic acid molecules to form a circular double-stranded nucleic acid construct for use in transfection, electroporation, nucleofection, and/or other form of delivery of the circular nucleic acid construct into a eukaryotic cell, of the present invention may comprise one, two, three, four, five, six, seven, eight, nine, ten, 20, 30, 40, 50, 60, 70, 80, 90, 100, 200, 300, 400, 500, 600, 700, 800, 900, 1000 or more individual linear double-stranded nucleic acid molecules in the mixture for joining (ligating) to form a circular double-stranded nucleic acid construct (nanocircle) of the present invention. In some embodiments, one linear double-stranded nucleic acid may be joined to itself, a process referred to as self-ligation. In some embodiments, two or more linear double-stranded nucleic acid molecules may be joined. In some embodiments, the two or more linear double-stranded nucleic acid molecules may comprise wherein at least one of the nucleic acid molecules is a “backbone” construct also referred to herein as a nanobackbone, and the other at least one nucleic acid molecules is an “insert” comprising and/or encoding a product (e.g., a gene or gene product, e.g., a nucleic acid and/or protein). In some embodiments, the nanobackbone may comprise and/or encode a product.

The cell for transfer of the produced circular double-stranded nucleic acid constructs, also referred to as nanocircles, may be any type of cell. In some embodiments, the cell is a eukaryotic cell. In some embodiments, the cell is a mammalian cell. In some embodiments, the cell is a human cell. In some embodiments, the cell is an immortalized cell line (e.g., a human cell line, e.g., including but not limited to HeLa cells, HEK293T cells, and the like). In some embodiments, the cell is an in vitro cell. In some embodiments, the cell is an ex vivo cell, e.g., a cell isolated from a human patient, e.g., a cell isolated from a sample of a human patient (e.g., a biopsy sample, a blood sample, a mucosal swab sample, and the like).

The exonuclease contemplated for use in the methods of the invention may be any 5′ to 3′ exonuclease that lacks the 3′ to 5′ exonuclease activity and whose 5′ to 3′ exonuclease hydrolysis activity is reduced by the modified phosphodiester group of each of the linear double-stranded nucleic acid molecules. In some embodiments, the exonuclease may be T7 DNA exonuclease.

The DNA ligase may be any DNA ligase which can join the sticky ends produced by the any 5′ to 3′ exonuclease that lacks the 3′ to 5′ exonuclease activity and whose 5′ to 3′ exonuclease hydrolysis activity is reduced by the modified phosphodiester group of each of the linear double-stranded nucleic acid molecules. Non-limiting examples of the DNA ligase include T7 DNA ligase, Taq DNA ligase and/or T4 DNA ligase. In some embodiments, the DNA ligase is T7 DNA ligase.

In some embodiments, the one or more linear double-stranded nucleic acid molecules to be joined, wherein each of nucleic acid molecules to be joined comprises overlapping terminal sequences flanked by one or more nucleotides connected via a modified phosphodiester group, may comprise one, two, three, four, five, six, seven, eight, nine, or ten immediately adjacent nucleotides connected via one, two, three, four, five, six, seven, eight, nine, or ten modified phosphodiester groups. While not wishing to be bound to theory, the phosphodiester modifications define precise “stop” positions for 5′-3′ DNA hydrolytic activity of the T7 DNA Exonuclease. This results in formation of single-stranded 3′-extended homology regions that anneal to each other, connecting terminal homology regions of a single DNA fragment or terminal homology regions of different DNA fragments in the desired order and orientation.

In some embodiments, the modified phosphodiester group(s) is a phosphorothioate group.

The single reaction mixture of the invention may optionally comprise additional components which may provide additional benefit. For example, in some embodiments, the single reaction mixture may be supplemented with a DNA polymerase. Non-limiting examples of a DNA polymerase of use to the invention is a Sulfolobus DNA Polymerase IV (Dpo4) and/or any other DNA polymerase that lacks the 5′ to 3′ exonuclease activity, 3′ to 5′ exonuclease activity, and strand displacement activity.

Furthermore, while no additional treatments, clean-ups, bacterial cloning and/or nanocircle or product amplification is required, in some embodiments, the invention may optionally comprise additional treatments which may provide additional benefit. For example, in some embodiments, the single reaction mixture may be treated with a T5 exonuclease after initial reaction is allowed to occur.

No bacterial sequence elements are required in the constructs of the present invention, though they may be optionally included. For example, in some embodiments, the circularized nucleic acid construct formed by the one or more joined linear nucleic acid molecules are devoid of bacterial sequence elements, such as bacterial antibiotic resistance marker(s) and/or bacterial origin(s) of replication. In some embodiments, the circularized nucleic acid construct formed by the one or more joined linear nucleic acid molecules may optionally comprise one or more bacterial sequence elements, such as bacterial antibiotic resistance marker(s) and/or bacterial origin(s) of replication.

linear DNA molecule(s) which are joined and/or circularized to produce DNA molecules defined as circular double-stranded DNA molecules devoid of bacterial DNA sequence elements such as bacterial antibiotic resistance marker(s) and/or bacterial origin(s) of replication may also be referred to as “DNA minicircles” or “minicircles”.

The joining reaction is performed for a time period and under conditions suitable for joining the one or more linear double-stranded nucleic acid molecules using concomitant activity of the 5′ to 3′ exonuclease and the DNA ligase in the single reaction. In some embodiments, the single reaction mixture is incubated for a time period of about 15 minutes to about 1 hour, e.g., about 15, 16, 17, 18, 19, 20, 21, 22, 23, 24, 25, 26, 27, 28, 29, 30, 31, 32, 33, 34, 35, 36, 37, 38, 3, 40, 41, 42, 42, 43, 44, 45, 46, 47, 48, 49, 50, 51, 52, 53, 54, 55, 56, 57, 58, 59 or 60 minutes (1 hour) or any value or range therein. For example, in some embodiments, the single reaction mixture may be incubated for a time period of about 15 minutes to about 60 minutes, about 20 minutes to about 40 minutes, about 30 minutes to about 40 minutes, or about 25 minutes to about 45 minutes. In some embodiments, the single reaction mixture may be incubated at a temperature of about 20° C. to about 30° C., e.g., about 20, 21, 22, 23, 24, 25, 26, 27, 28, 29, or 30° C. or any value or range therein. For example, in some embodiments, the single reaction mixture may be incubated at a temperature of about 20° C. to about 27° C., about 22.5° C. to about 30° C., or about 22° C., about 23° C., about 24° C., about 24.5° C., about 25° C., about 25.5° C., about 26° C., about 27° C., about 28° C., about 29° C., or about 30° C.

The method of the invention may further be devoid of or comprise any one of the following steps: DNA restriction digest of the synthetic nucleic acid molecule comprising the inserted heterologous polynucleotide and/or the circular synthetic nucleic acid molecule not comprising an insert, utilization of bacterial (e.g., E. coli) competent cells, transformation of the synthetic nucleic acid molecule comprising the inserted heterologous polynucleotide into bacterial cells (e.g., E. coli), amplification of the synthetic nucleic acid molecule comprising the inserted heterologous polynucleotide in bacteria (e.g., plating of transformed bacteria, bacterial colony picking, growth in liquid or solid cultures of bacteria transformed with the synthetic nucleic acid molecule comprising the inserted heterologous polynucleotide, endotoxin removal procedures, or any combination thereof. In some embodiments, the method of the invention is devoid of each of the above steps.

In some embodiments, the method of the present invention may further comprise, following incubation, isolating the circular nucleic acid construct from the single reaction mixture.

In some embodiments, the method of the present invention may further comprise providing the circular nucleic acid construct to the eukaryotic cell. In some embodiments, the method of the present invention may further comprise providing the circular nucleic acid construct to the eukaryotic cell in repeated doses, e.g., once, twice, three, four, five, six, seven, eight, nine, or ten times or more, e.g., serially.

Also provided herein are methods of making the nanocomponents for use in the method of joining of the invention. Accordingly, another aspect of the present invention provides a method of producing a synthetic linear double-stranded nucleic acid molecule comprising overlapping terminal sequences flanked by one or more nucleotides connected via a modified phosphodiester group (e.g., generating a linear double-stranded nucleic acid molecule for use in the method of joining of the invention; e.g., generating a “nano-backbone”), comprising: amplifying (e.g., PCR amplifying) a synthetic linear double-stranded nucleic acid molecule comprising an RNA polymerase terminator sequence, RNA cleavage and polyadenylation sequence, and/or an RNA scaffold followed in 5′ to 3′ orientation by a promoter and/or an enhancer, to produce a population of synthetic linear double-stranded nucleic acid molecules under conditions in which each of the amplified linear double-stranded nucleic acid molecule generated comprises the overlapping terminal sequences flanked by the one or more nucleotides connected via a modified phosphodiester group.

While not wishing to be bound to theory, the typical order of upstream and downstream sequences for bacteria-propagated plasmids is reversed in the nanotemplates of the present invention in such a way that a typical downstream sequence, such as an RNA polymerase terminator sequence, RNA cleavage and polyadenylation sequence, and/or an RNA scaffold, is now followed by a typical upstream sequence, such as sequence of a promoter and/or an enhancer, and wherein the said nano-templates serve as templates for polymerase chain reactions (PCRs) to produce linear DNA molecules (“nano-backbones”) that comprise terminal homology sequences flanked by nucleotides connected via modified phosphodiester groups that are requisite for the method of joining of the present invention.

Accordingly, also provided herein is a synthetic double-stranded nucleic acid molecule (e.g., “nano-template”) comprising an RNA polymerase terminator sequence, RNA cleavage and polyadenylation sequence, and/or an RNA scaffold followed in 5′ to 3′ orientation by a promoter and/or an enhancer, for use in the methods of the present invention.

While not wishing to be bound to theory, the produced nanobackbones of these nanotemplates also comprise wherein the typical plasmid order of upstream and downstream sequences is reversed in such a way that a typical downstream sequence, such as an RNA polymerase terminator sequence, RNA cleavage and polyadenylation sequence, and/or an RNA scaffold, is now followed by a typical upstream sequence, such as sequence of a promoter and/or an enhancer, wherein the nanobackbones also comprise terminal homology sequences flanked by nucleotides connected via modified phosphodiester groups that are requisite for the methods of joining of the present invention. In some embodiments, said nano-backbones may also comprise or lack bacterial DNA sequences such as but not limited to those encoding antibiotic resistance marker(s) and/or origin(s) of replication.

Accordingly, another aspect of the present invention provides a synthetic linear double-stranded nucleic acid molecule (e.g., “nano-backbone”) comprising: an RNA polymerase terminator sequence, RNA cleavage and polyadenylation sequence, and/or an RNA scaffold followed in 5′ to 3′ orientation by a promoter and/or an enhancer, and the overlapping terminal sequences flanked by the one or more nucleotides connected via a modified phosphodiester group produced by the methods of the present invention and/or for use in the methods of the present invention.

Also provided herein is a kit comprising one or more components of the present invention such as the nanocircles, nanobackbones, nanotemplates, mixture reagents, PCR primers, and the like, and optional instructions for the use thereof.

Kits that include nucleic acid molecules and/or nucleic acid constructs of this invention and/or a pharmaceutical composition as described herein are also provided herein. Some kits include components in a container (e.g., vial or ampule), and may also include instructions for use of the components in the various methods disclosed above. The components can be in various forms, including, for instance, as part of a solution or as a solid (e.g., lyophilized powder). The instructions may include a description of how to prepare (e.g., dissolve or resuspend) the components in an appropriate fluid and/or how to administer the components for the treatment of the diseases and disorders described herein.

The kits may also include various other components, such as buffers, salts, complexing metal ions and other agents as needed, e.g., for transport stability, for PCR reactions, etc. These components may be included with the components or may be in separate containers.

In some embodiments, the present invention may be as defined in any one of the following numbered paragraphs.
1. An in vitro method for joining ends of one or more linear double-stranded nucleic acid molecules to form a circular double-stranded nucleic acid construct for use in transfection, electroporation, nucleofection, and/or other form of delivery of the circular nucleic acid construct into a eukaryotic cell, comprising: joining the ends of the one or more linear nucleic acid molecules in a single reaction comprising the following mixture of components (a) to (d): (a) the one or more linear double-stranded nucleic acid molecules to be joined, wherein each of nucleic acid molecules to be joined comprises ends (e.g., a 5′ end and a 3′ end) comprising overlapping terminal sequences flanked by one or more nucleotides connected via a modified phosphodiester group, (b) a 5′ to 3′ exonuclease that lacks the 3′ to 5′ exonuclease activity and whose 5′ to 3′ exonuclease hydrolysis activity is reduced by the modified phosphodiester group of each of the linear double-stranded nucleic acid molecules, (c) a DNA ligase, and (d) a buffer suitable for joining the ends of said one or more linear double-stranded nucleic acid molecules using concomitant activity of the 5′ to 3′ exonuclease and the DNA ligase in the single reaction; wherein at least one of the one or more linear nucleic acid molecules comprises and/or encodes a product for expression in the eukaryotic cell, and wherein the product is expressed in the eukaryotic cell upon delivery without the requirement to transform and/or propagate the circular nucleic acid construct in bacteria at any step of the method.
2. The method of claim paragraph 1, wherein the 5′ to 3′ exonuclease and the DNA ligase act sequentially.
3. The method of paragraphs 1 or 2, comprising two or more (e.g., two or more, three or more, four or more, 10 or more, 100 or more, etc.) linear double-stranded nucleic acid molecules in the mixture.
4. The method of any one of paragraphs 1-3, wherein the eukaryotic cell is a mammalian cell.
5. The method of any one of paragraphs 1-4, wherein the eukaryotic cell is a human cell.
6. The method of any one of paragraphs 1-5, wherein the eukaryotic cell is an in vitro or ex vivo cell.
7. The method of any one of paragraphs 1-6, wherein the exonuclease of (b) is T7 DNA exonuclease.
8. The method of any one of paragraphs 1-7, wherein the ligase of (c) is T7 DNA ligase, Taq DNA ligase and/or T4 DNA ligase.
9. The method of any one of paragraphs 1-8, wherein the ligase of (c) is T7 DNA ligase.
10. The method of any one of paragraphs 1-9, wherein the one or more nucleotides connected via a modified phosphodiester group comprises two, three, four, five, six, seven, eight, nine, or ten immediately adjacent nucleotides connected via modified phosphodiester groups.
11. The method of any one of paragraphs 1-10, wherein the modified phosphodiester group(s) is a phosphorothioate group.
12. The method of any one of paragraphs 1-11, wherein the single reaction mixture is supplemented with a DNA polymerase.
13. The method of paragraph 12, wherein the DNA polymerase is a Sulfolobus DNA Polymerase IV (Dpo4) and/or any other DNA polymerase that lacks the 5′ to 3′ exonuclease activity, 3′ to 5′ exonuclease activity, and strand displacement activity.
14. The method of any one of paragraphs 1-13, wherein the single reaction mixture is treated with a T5 exonuclease after initial reaction is allowed to occur.
15. The method of any one of paragraphs 1-14, wherein the circularized nucleic acid construct formed by the two or more joined linear nucleic acid molecules are devoid of bacterial sequence elements.
16. The method of paragraph 15, wherein the circularized nucleic acid construct formed by the two or more joined linear nucleic acid molecules are devoid of bacterial antibiotic resistance marker(s) and/or bacterial origin(s) of replication.
17. The method of any one of paragraphs 1-16, wherein the single reaction mixture is incubated for a time period of about 15 minutes to about 1 hour.
18. The method of any one of paragraphs 1-17, wherein the single reaction mixture is incubated at a temperature of about 20° C. to about 30° C.
19. The method of any one of paragraphs 1-18, wherein the single reaction mixture is incubated at a temperature of about room temperature (about 25° C.).
20. The method of any one of paragraphs 1-19, further comprising a step of performing a clean-up procedure).
21. The method of any one of paragraphs 1-20, further comprising, following incubation, isolating the circular nucleic acid construct from the single reaction mixture.
22. The method of any one of paragraphs 1-21, further comprising providing the circular nucleic acid construct to the eukaryotic cell.
23. A method of producing a synthetic linear double-stranded nucleic acid molecule comprising overlapping terminal sequences flanked by one or more nucleotides connected via a modified phosphodiester group (e.g., generating a linear double-stranded nucleic acid molecule for use in any one of paragraphs 1-22; e.g., generating a “nano-backbone”), comprising: amplifying (e.g., PCR amplifying) a synthetic linear double-stranded nucleic acid molecule comprising an RNA polymerase terminator sequence, RNA cleavage and polyadenylation sequence, and/or an RNA scaffold followed in 5′ to 3′ orientation by a promoter and/or an enhancer, to produce a population of synthetic linear double-stranded nucleic acid molecules under conditions in which each of the amplified linear double-stranded nucleic acid molecule generated comprises the overlapping terminal sequences flanked by the one or more nucleotides connected via a modified phosphodiester group.
24. A synthetic double-stranded nucleic acid molecule (e.g., “nano-template”) comprising an RNA polymerase terminator sequence, RNA cleavage and polyadenylation sequence, and/or an RNA scaffold followed in 5′ to 3′ orientation by a promoter and/or an enhancer, for use in paragraph 23.
25. A synthetic linear double-stranded nucleic acid molecule (e.g., “nano-backbone”) comprising: an RNA polymerase terminator sequence, RNA cleavage and polyadenylation sequence, and/or an RNA scaffold followed in 5′ to 3′ orientation by a promoter and/or an enhancer, and the overlapping terminal sequences flanked by the one or more nucleotides connected via a modified phosphodiester group produced by the method of paragraph 23.
26. A synthetic linear double-stranded nucleic acid molecule (e.g., “nano-backbone”) comprising: an RNA polymerase terminator sequence, RNA cleavage and polyadenylation sequence, and/or an RNA scaffold followed in 5′ to 3′ orientation by a promoter and/or an enhancer, and overlapping terminal sequences flanked by one or more nucleotides connected via a modified phosphodiester group, for use in the method of any one of paragraphs 1-22.
27. The method of any one of paragraphs 1-26, wherein the product of the reaction is linear.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1

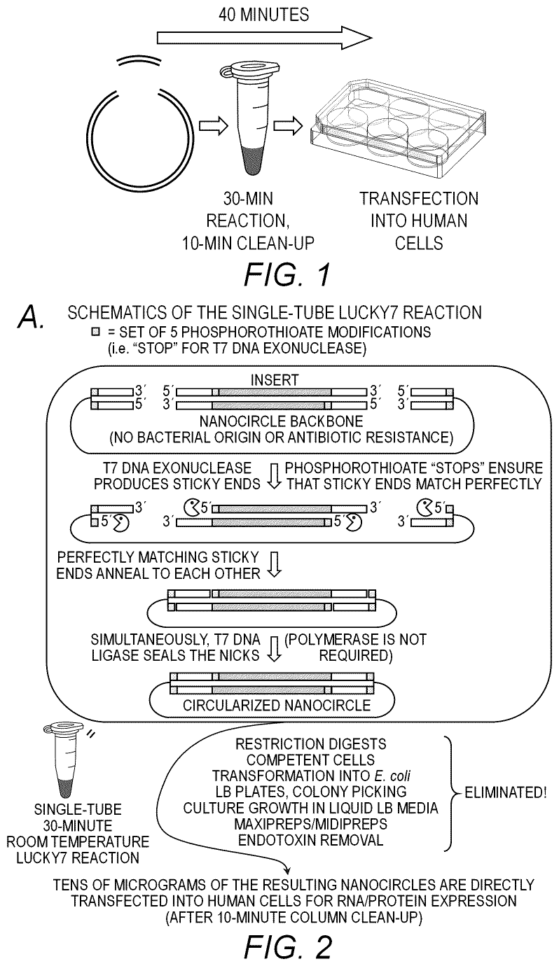

The E. coli-free approach of the present invention employs the joining of one or more double-stranded DNA molecules in a single, brief, room temperature reaction, enabling the direct use of the resulting reaction products in human cells for efficient expression of RNAs and/or proteins (FIG. 1).

A major advantage of this approach is its high yield, which eliminates the need for traditional amplification of the reaction products in bacteria prior to the use in human cells.

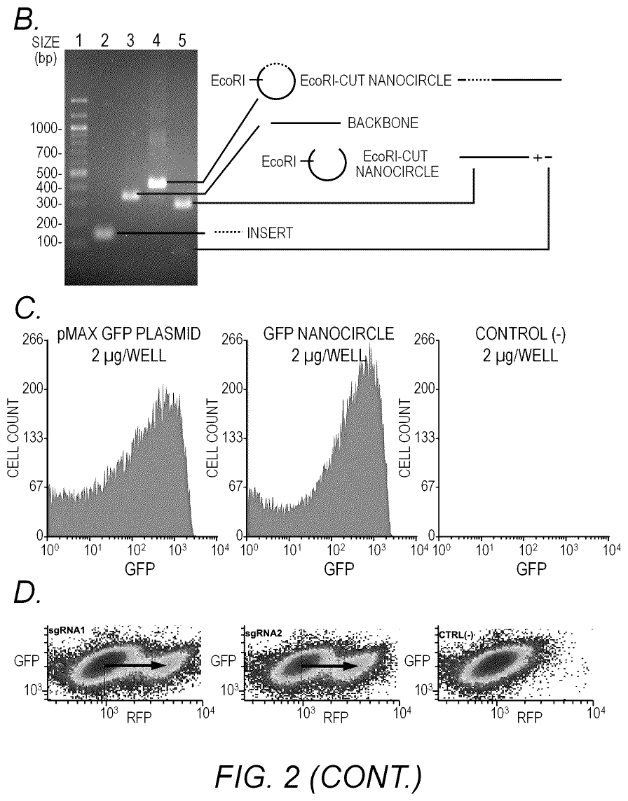

Due to elimination of bacterial steps, the method disclosed herein shortens the entire process of DNA joining and preparation for use in eukaryotic cells such as human cells from several days to about 40 minutes. Once transfected into eukaryotic cells, the joined DNA molecules efficiently express proteins and RNAs, as demonstrated using an array of fluorescent proteins and antibiotic selection markers as well as CRISPR-based knockouts and shRNA-based knockdowns of targeted human genes, as further described in FIGS. 2-6.

In brief, FIG. 2 provides a schematic workflow of the method described herein (FIG. 2 panel A), the components thereof (FIG. 2 panel B). Unlike in assembly methods relying on other exonucleases such as T5, the T7 exonuclease does not degrade the nucleic acid molecules (nanobackbone or insert) past the phosphorothioate modifications, result in near-complete annealing and ligation (“joining”) of the nucleic acid molecules. FIG. 2 panel C further provides data evidence showing successful product expression as shown by fluorescence signal produced in HEK293T cells after transfection of 2 μg of GFP nanocircles produced by the methods disclosed herein and immediately transferred to the HEK293T cells (FIG. 2 panel C, middle). This fluorescence signature of the nanocircles by the methods described herein (i.e., assembled and immediately transferred to a eukaryotic cell without intervening steps of bacterial amplification and the like) resulted in higher fluorescence due to their small size (lacking ori and AmpR, as compared to a standard pMaxGFP plasmid (FIG. 2 panel C; left). Similarly, FIG. 2 panel D shows that transfection of sgRNA-expressing nanocircles produced efficient knockout of the NMD factor CASC3 in Cas9-expressing Fireworks HeLa cells as compared to control.

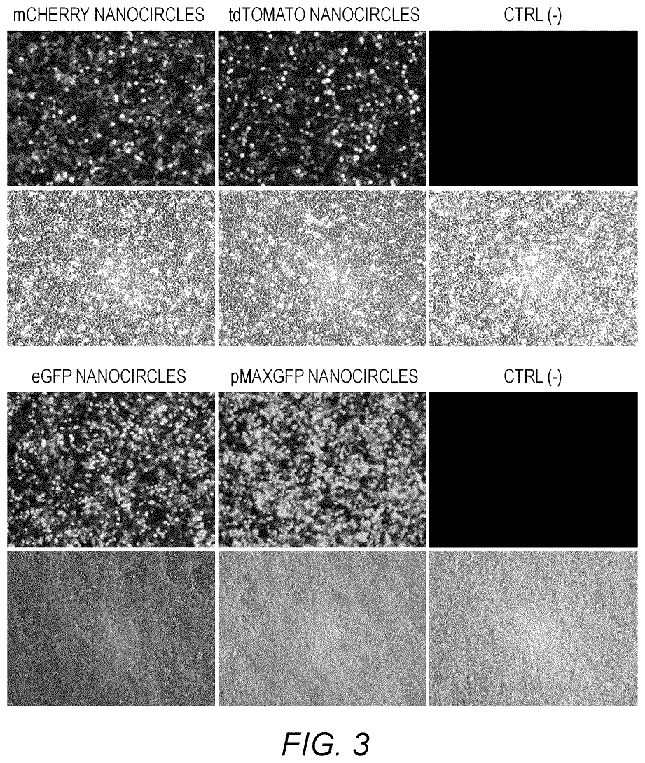

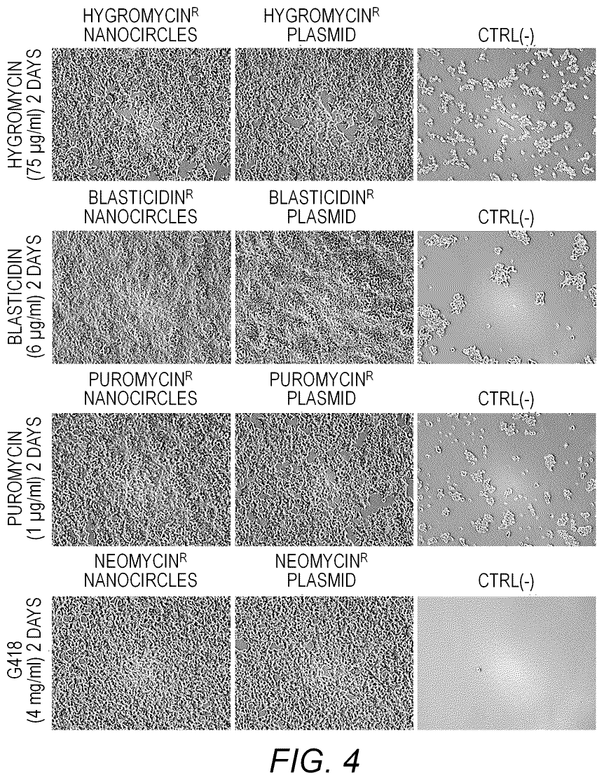

In addition, FIG. 3 and FIG. 4 show efficient transfection and expression of fluorescent proteins and functionally-active antibiotic selection markers by enzymatically-produced DNA nanocircles in human cells. To further highlight the generality of this approach, we demonstrate that lentiviruses constructed using the methods described herein efficiently integrate into the human genome and produce robust gene inactivation.

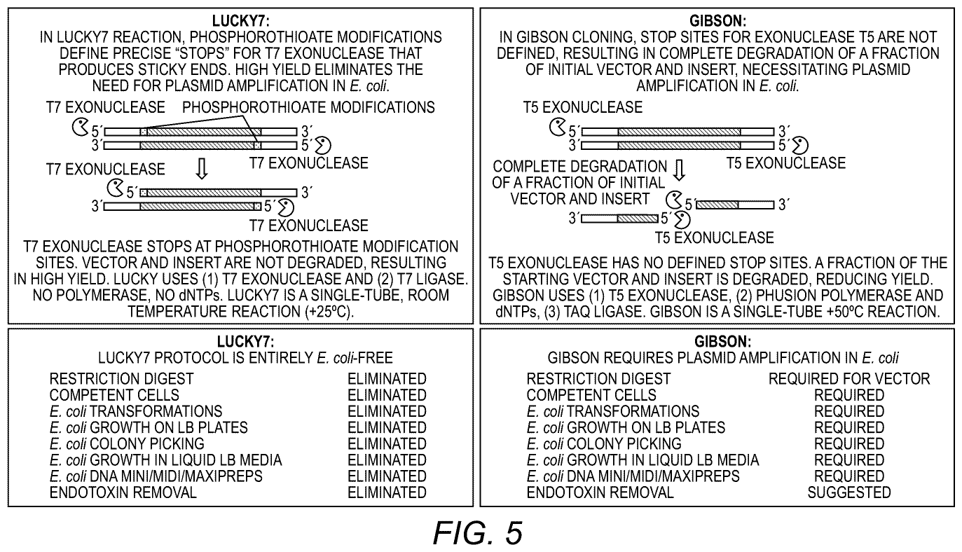

The approach of the methods disclosed herein combines two activities in a single 30-minute, room temperature reaction: the 5′-exonucleolytic activity of T7 DNA Exonuclease and the nick-sealing activity of T7 DNA ligase (FIG. 5). In this reaction, precisely positioned phosphorothioate DNA modifications serve as stops for T7 DNA Exonuclease, producing fully defined perfectly matching single stranded sticky ends for annealing of a DNA insert to nano-backbone. Simultaneously with the process of sticky end formation and annealing, T7 DNA Ligase seals the newly formed insert-backbone nicks. Following a 10-minute column clean-up, the reaction products are ready for use in human cells.

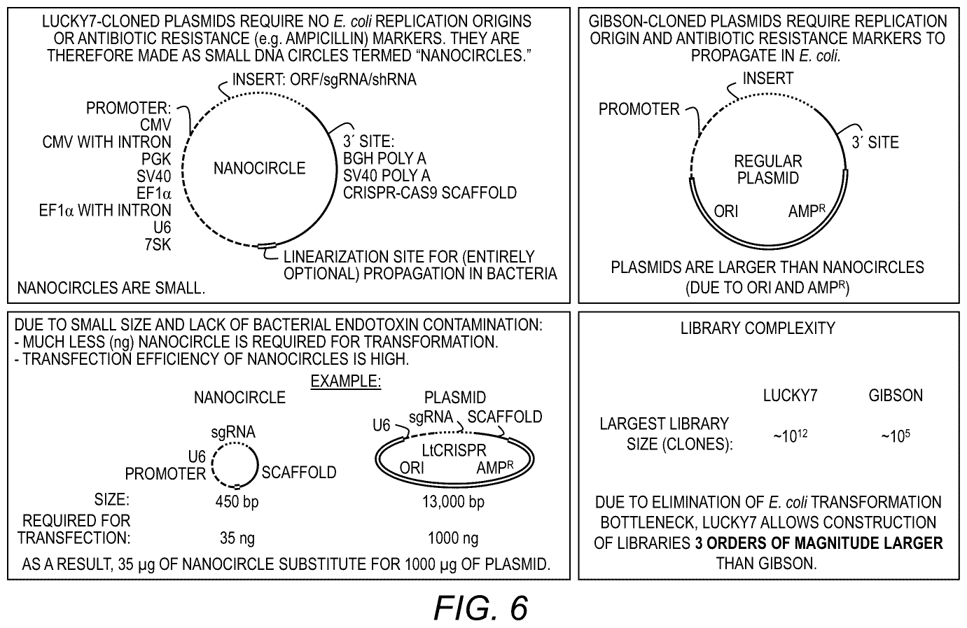

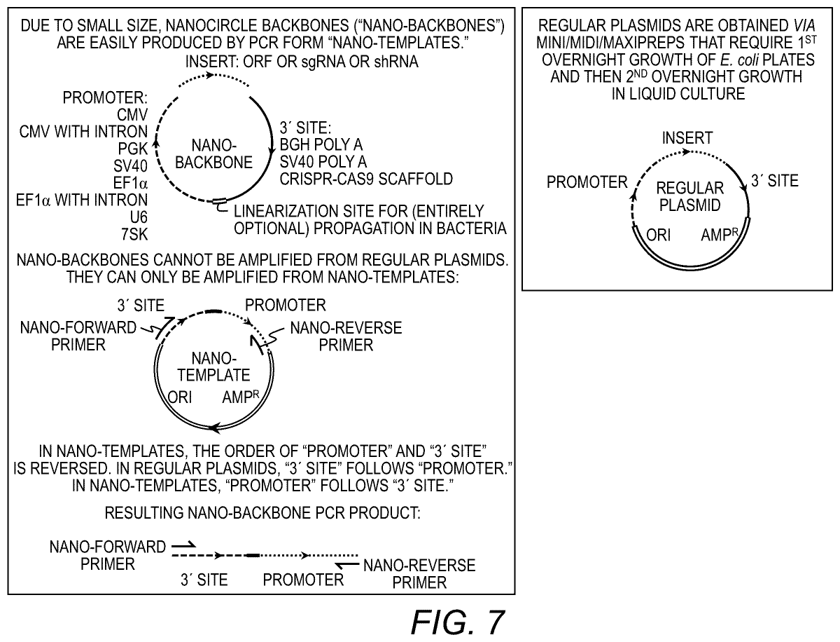

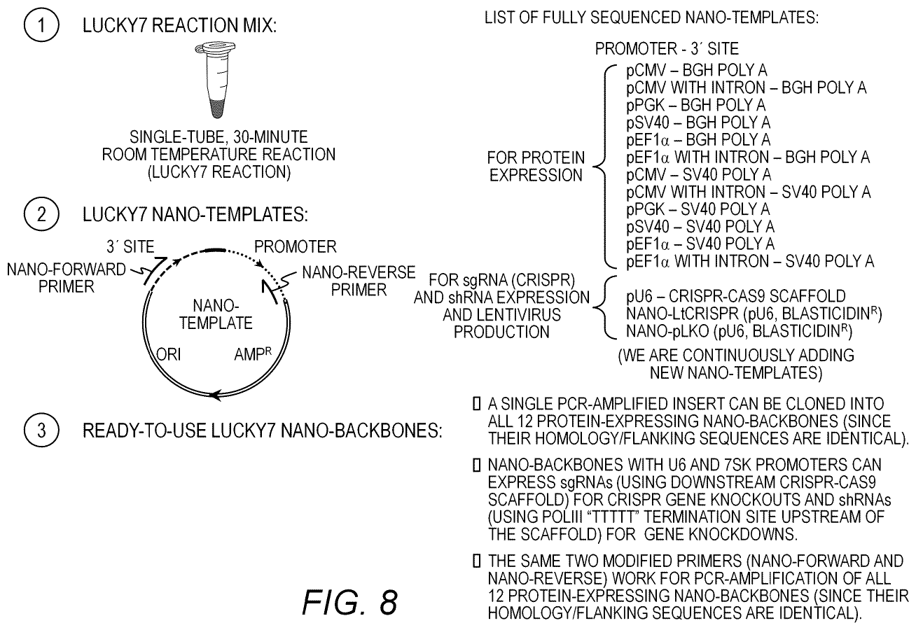

Due to the elimination of E. coli from the procedure, the nano-backbones used in the method do not require a bacterial origin of replication or antibiotic resistance marker. To express an RNA or protein in human cells, nano-backbones only need to encode an RNA Polymerase III termination or RNA Polymerase II cleavage and polyadenylation sequence, followed by a promoter (FIGS. 6 and 7). This order is atypical for traditional DNA plasmids, in which the promoter typically precedes the terminator and/or RNA cleavage and polyadenylation sequence. The resulting small size and absence of bacterial endotoxin in the method of the present invention contribute to high transfection efficiency of the produced nano-circles in human cells; their efficiency rival or surpasses that of conventional bacteria-derived plasmids. The nanotemplates and nanobackbones can and have been generated in a variety of formats comprising variable combinations of RNA Polymerase III termination or RNA Polymerase II cleavage and polyadenylation sequences, and promoters (FIG. 8).

In comparison to conventional E. coli-based DNA cloning methods, the methods of the present invention require none of the following procedures: DNA restriction digest, utilization of E. coli, competent cells, transformation of DNA into E. coli, plating of transformed bacteria on LB plates followed by their overnight incubation, bacterial colony picking, secondary overnight growth of bacterial cultures in liquid LB media, bacterial culture centrifugation, mini/midi/maxipreps of plasmid from pelleted bacteria, and endotoxin removal from the bacteria-derived plasmid DNA. As a result, this approach yields savings of time, labor, and resources, and enables rapid expression of RNA and proteins in human cells.

### Example 2

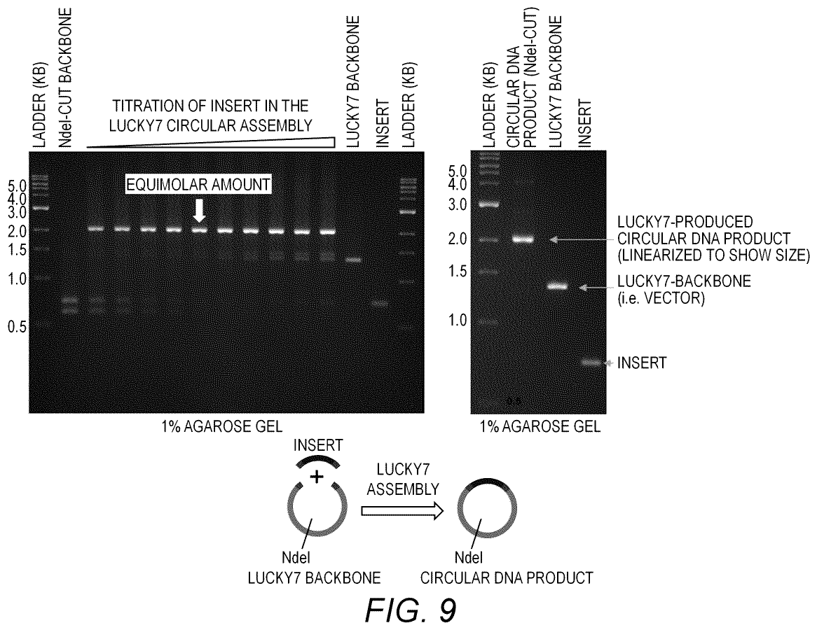

FIG. 9 shows an example of a two-piece Lucky7 assembly of a 0.7 kb DNA insert into a 1.4 kb DNA backbone (vector) to illustrate that their near-complete assembly into the circular DNA product occurs when the insert and the backbone are added to the Lucky7 reaction in equimolar (stoichiometric) amounts. The sample in which the insert and the backbone were added in equimolar amounts is indicated by a white arrow.

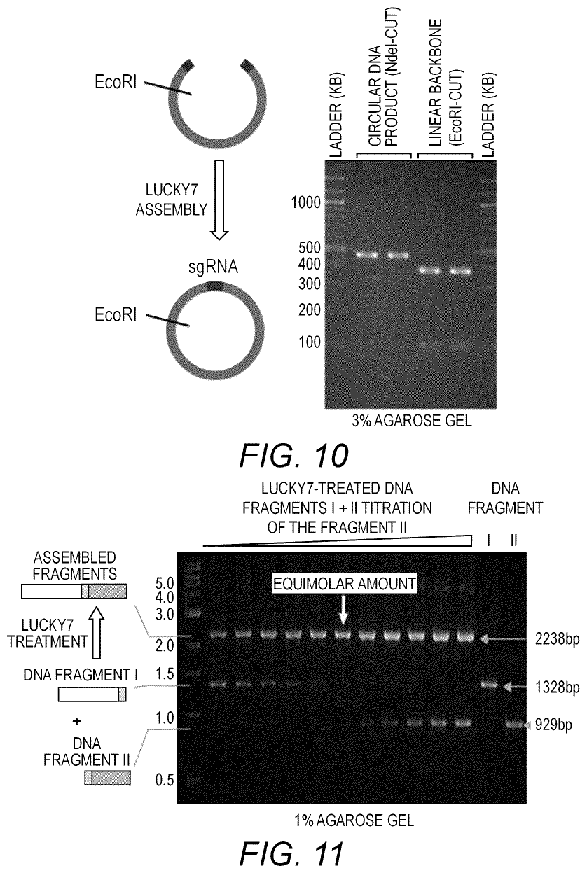

FIG. 10 shows an example of circularization of a linear DNA using the Lucky7 assembly, illustrating a simple, rapid, and efficient production of sgRNA-expressing DNA nanocircles for CRISPR-based knockouts and mutagenesis of human genes. An agarose gel displays circularized nanocircles encoding two different sgRNAs, as well as their uncircularized Lucky7-untreated linear double-stranded DNA precursors.

FIG. 11 shows an example of Lucky7 assembly of two linear DNA fragments into a single DNA product. Similar to the assembly of two linear DNA products into a circular product shown in FIG. 9, near-complete assembly of linear products occurs here when the fragments are added to the Lucky7 reaction in equimolar (stoichiometric) amounts. The sample in which fragment I and fragment II were added in equimolar amounts is indicated by a white arrow).

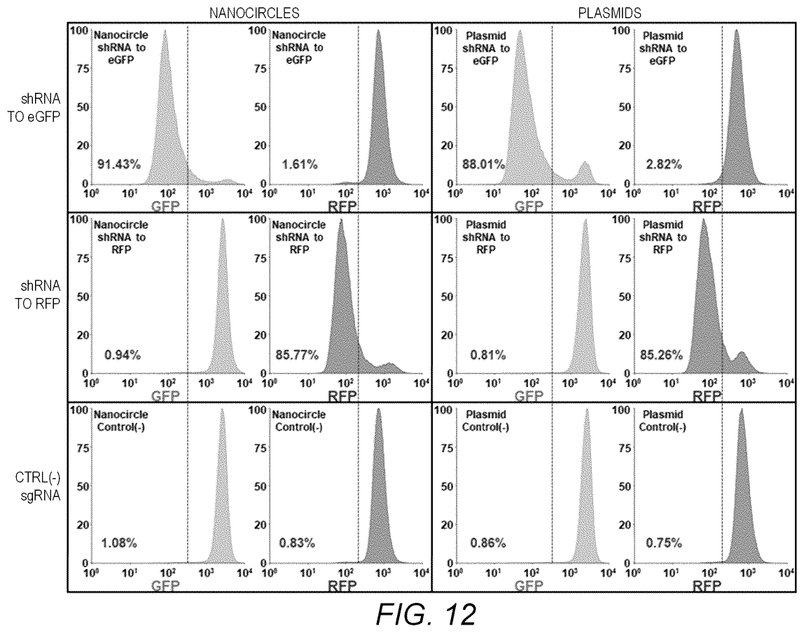

FIG. 12 shows an example of using Lucky7-assembled shRNA-expressing nanocircles to downregulate GFP and RFP stably expressed in a HeLa cell line. In this experiment, shRNA-expressing nanocircles targeting GFP and RFP were transfected into human cells, and their silencing efficiency for GFP and RFP was analyzed by FACS and compared with that of shRNAs expressed by a similarly transfected pLKO.1 plasmid.

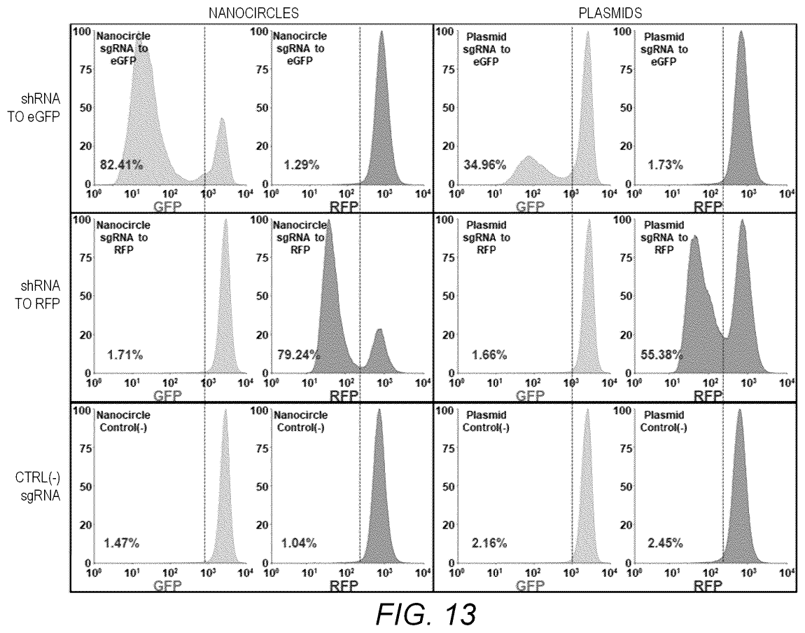

FIG. 13 shows an example of using Lucky7-assembled sgRNA-expressing nanocircles to knockout GFP and RFP stably expressed in a HeLa cell line that also expressed Cas9. In this experiment, sgRNA-expressing nanocircles targeting GFP-and RFP-expressing genes were transfected into human cells, and their knockout efficiency for GFP and RFP was analyzed by FACS and compared with that of sgRNAs expressed by a similarly transfected LentiCRISPR plasmid.

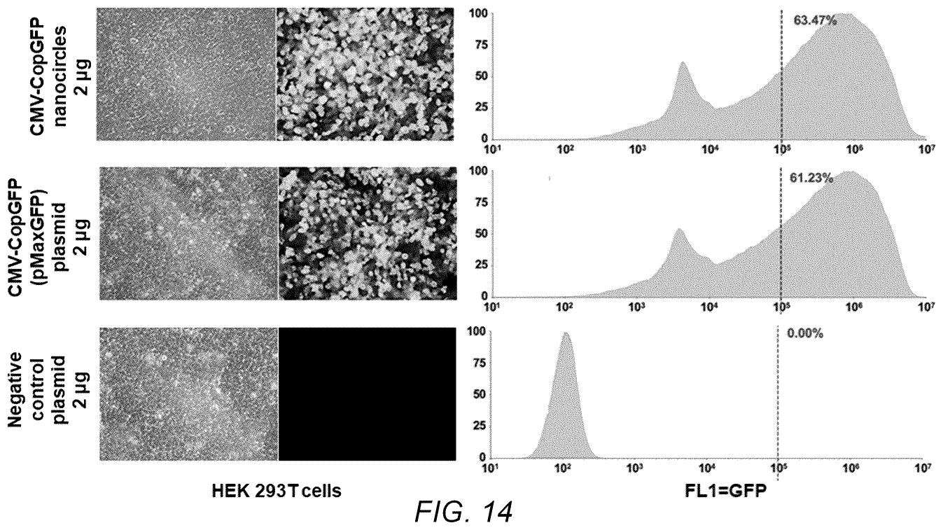

FIG. 14 shows microscopy and FACS analysis of green fluorescence produced by Lucky7-assembled CopGFP-expressing nanocircles transiently transfected into human HEK293T cells. In this figure, nanocircle-produced fluorescence of CopGFP is directly compared with that produced by a CopGFP-expressing plasmid, pMaxGFP, of which an identical amount (2 micrograms) was similarly transfected into HEK293T cells. CopGFP is a green fluorescent protein isolated from the copepod Pontellina plumata.

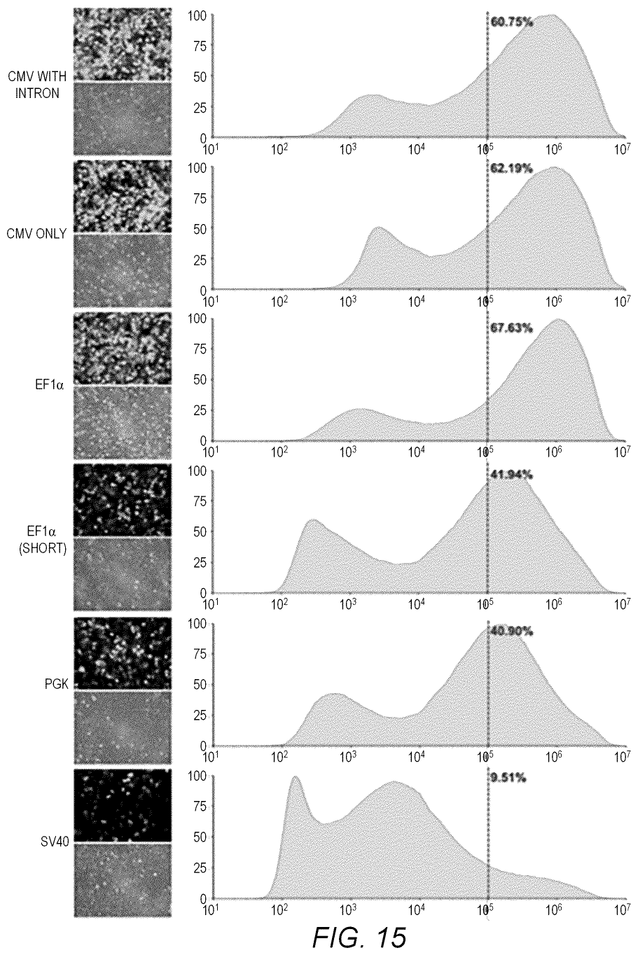

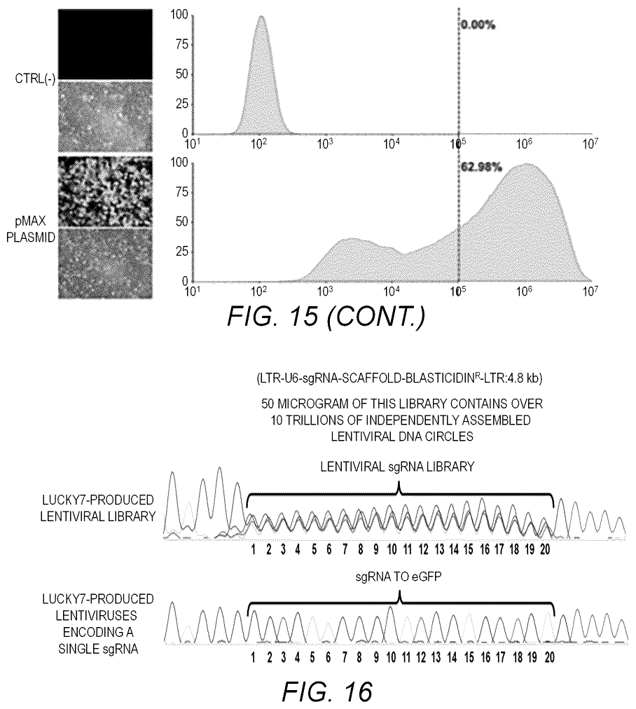

FIG. 15 shows microscopy and FACS analysis of fluorescence produced in human HEK293T cells by transiently transfected nanocircles expressing CopGFP under the control of the following promoters: CMV with intron, CMV, EF1α, EF1α(short), PGK, and SV40. The fluorescence of a similarly transfected CopGFP-expressing control plasmid, pMaxGFP, is shown as a control.

FIG. 16 shows sequencing traces for a single sgRNA that targets GFP and a pool of 62,000 sgRNAs assembled into a lentiviral backbone using Lucky7 assembly. The resulting circular lentiviral DNA library is 4.8 kb large; it consists of an LTR-pU6-sgRNA-scaffold-pEF1α(short)-BlasticidinR-LTR backbone and expresses sgRNAs and sgRNA libraries under the control of the U6 promoter.

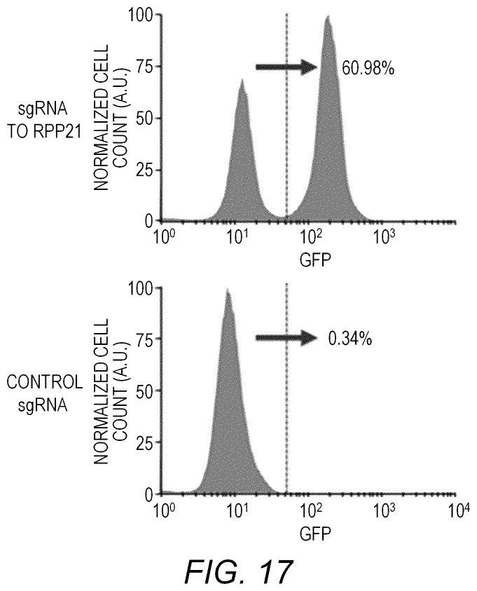

FIG. 17 shows a 10-fold increase in GFP fluorescence in over 60% of cells in the Mirror HeLa cell line. It results from knockout of an endogenous human gene, RPP21, by Lucky7-assembled lentiviruses that express sgRNA targeting RPP21. In this cell line, an increase in green fluorescence signifies inhibition of Ribonuclease P activity, triggered by inactivation of RPP21.

The foregoing is illustrative of the present invention, and is not to be construed as limiting thereof. The invention is defined by the following claims, with equivalents of the claims to be included therein.

What is claimed is:

1. An in vitro method for joining ends of one or more linear double-stranded nucleic acid molecules to form a circular double-stranded nucleic acid construct for use in transfection, electroporation, nucleofection, and/or other form of delivery of the circular nucleic acid construct into a eukaryotic cell, comprising:
joining the ends of the one or more linear nucleic acid molecules in a single reaction comprising the following mixture of components (a) to (d):
(a) the one or more linear double-stranded nucleic acid molecules to be joined, wherein each of nucleic acid molecules to be joined comprises ends comprising overlapping terminal sequences flanked by one or more nucleotides connected via a modified phosphodiester group,
(b) a 5′ to 3′ exonuclease that lacks the 3′ to 5′ exonuclease activity and whose 5′ to 3′ exonuclease hydrolysis activity is reduced by the modified phosphodiester group of each of the linear double-stranded nucleic acid molecules,
(c) a DNA ligase, and
(d) a buffer suitable for joining the ends of said one or more linear double-stranded nucleic acid molecules using concomitant activity of the 5′ to 3′ exonuclease and the DNA ligase in the single reaction;
wherein at least one of the one or more linear nucleic acid molecules comprises and/or encodes a product for expression in the eukaryotic cell, and wherein the product is expressed in the eukaryotic cell upon delivery without the requirement to transform and/or propagate the circular nucleic acid construct in bacteria at any step of the method.

2. The method of claim 1, wherein the 5′ to 3′ exonuclease and the DNA ligase act sequentially.

3. The method of claim 1, comprising two or more linear double-stranded nucleic acid molecules in the mixture.

4. The method of claim 1, wherein the eukaryotic cell is a mammalian cell.

5. The method of claim 1, wherein the eukaryotic cell is a human cell.

6. The method of claim 1, wherein the eukaryotic cell is an in vitro or ex vivo cell.

7. The method of claim 1, wherein the exonuclease of (b) is T7 DNA exonuclease.

8. The method of claim 1, wherein the ligase of (c) is T7 DNA ligase, Taq DNA ligase and/or T4 DNA ligase.

9. The method of claim 1, wherein the one or more nucleotides connected via a modified phosphodiester group comprises two, three, four, five, six, seven, eight, nine, or ten immediately adjacent nucleotides connected via modified phosphodiester groups.

10. The method of claim 1, wherein the modified phosphodiester group(s) is a phosphorothioate group.

11. The method of claim 1, wherein the single reaction mixture is supplemented with a DNA polymerase.

12. The method of claim 11, wherein the DNA polymerase is a Sulfolobus DNA Polymerase IV (Dpo4) and/or any other DNA polymerase that lacks the 5′ to 3′ exonuclease activity, 3′ to 5′ exonuclease activity, and strand displacement activity.

13. The method of claim 1, wherein the single reaction mixture is treated with a T5 exonuclease after initial reaction is allowed to occur.

14. The method of claim 1, wherein the circularized nucleic acid construct formed by the two or more joined linear nucleic acid molecules are devoid of bacterial sequence elements.

15. The method of claim 14, wherein the circularized nucleic acid construct formed by the two or more joined linear nucleic acid molecules are devoid of bacterial antibiotic resistance marker(s) and/or bacterial origin(s) of replication.

16. The method of claim 1, wherein the single reaction mixture is incubated for a time period of about 15 minutes to about 1 hour.

17. The method of claim 1, further comprising, following incubation, isolating the circular nucleic acid construct from the single reaction mixture.

18. The method of claim 1, further comprising providing the circular nucleic acid construct to the eukaryotic cell.

19. A method of producing a synthetic linear double-stranded nucleic acid molecule comprising overlapping terminal sequences flanked by one or more nucleotides connected via a modified phosphodiester group, comprising:
amplifying a synthetic linear double-stranded nucleic acid molecule comprising an RNA polymerase terminator sequence, RNA cleavage and polyadenylation sequence, and/or an RNA scaffold followed in 5′ to 3′ orientation by a promoter and/or an enhancer, to produce a population of synthetic linear double-stranded nucleic acid molecules under conditions in which each of the amplified linear double-stranded nucleic acid molecule generated comprises the overlapping terminal sequences flanked by the one or more nucleotides connected via a modified phosphodiester group.

20. A synthetic linear double-stranded nucleic acid molecule comprising:
an RNA polymerase terminator sequence, RNA cleavage and polyadenylation sequence, and/or an RNA scaffold followed in 5′ to 3′ orientation by a promoter and/or an enhancer, and
the overlapping terminal sequences flanked by the one or more nucleotides connected via a modified phosphodiester group produced by the method of claim 19, optionally wherein the product of the reaction is linear.

21. The method of claim 1, wherein the product of the reaction is linear.
